(19) United States
(12) Patent Application Publication
(10) Pub. No.: US 2020/0174021 A1
(43) Pub. Date: Jun. 4, 2020

(54) METHODS FOR DIAGNOSIS OF EARLY STAGE HEART FAILURE

(71) Applicant: Queensland University of Technology, Brisbane, Queensland (AU)

(72) Inventors: Chamindie Punyadeera, Brisbane (AU); Xi Xhang, Brisbane (AU); Benjamin Schulz, Brisbane (AU)

(21) Appl. No.: 16/636,403

(22) PCT Filed: Aug. 8, 2018
(86) PCT No.: PCT/AU2018/050827

§ 371 Date: Feb. 4, 2020

(30) Foreign Application Priority Data
Aug. 8, 2017 (AU) 2017903138

(51) Int. Cl. G01N 33/68; G01N 33/543

(52) U.S. Cl. CPC G01N 33/6893; G01N 2800/60; G01N 2800/325; G01N 33/54306

(57) ABSTRACT

The invention relates to methods for diagnosing the early stages of heart failure. The invention particularly relates to diagnosing class I and class II heart failure, based on the New York Heart Association (NYHA) classification system. The invention can also discriminate between healthy controls and heart failure patients in NYHA class III/IV.

### TECHNICAL FIELD

The present invention relates to methods for diagnosing the early stages of heart failure. The invention particularly relates to diagnosing class I and class II heart failure, based on the New York Heart Association (NYHA) classification system. The invention can also discriminate between healthy controls and heart failure patients in NYHA class III/IV.

### BACKGROUND ART

Heart failure occurs when the heart muscle is weakened such that it can no longer pump sufficient blood to meet a body's requirements for blood and oxygen. In other words, the heart cannot keep up with its workload. There are a number of compensation mechanisms that come into play during the early stages of heart failure, including enlargement, increase in muscle mass, and faster pumping. Without treatment and/or lifestyle changes, eventually the compensation mechanisms are no longer effective, and the person starts to experience symptoms of heart failure, such as fatigue and breathing problems.

In the early part of the 20^{th} century, there was no way to take measurements of cardiac function and therefore there was no consistency of diagnosis. The NYHA developed a classification system that is still used today in clinical descriptions of heart failure (The Criteria Committee of the New York Heart Association, 1994). According to the NYHA classification system, patients are placed in one of four categories, based on their limitations during physical activity, any limitations or symptoms during normal breathing and shortness of breath and/or angina.

The classification system is set out in Table 1.

TABLE 1
NYHA functional classification of heart failure

| NYHA Class | Symptoms |
|---|---|
| I | No symptoms and no limitation during ordinary physical activity, for |
| | example, no shortness of breath or angina when walking or climbing |
| | stairs |
| II | Mild symptoms, for example, mild shortness of breath and/or angina, |
| | and slight limitation during ordinary physical activity |
| III | Marked limitation in activity due to symptoms even when walking short |
| | distances (20-100 metres), comfortable only at rest |
| IV | Severe limitations, symptoms even when at rest, any physical activity |
| | increases discomfort |

Heart failure imposes substantial social and economic burdens on society, predominantly due to its high global prevalence. For example, it is estimated that 23 million people worldwide are diagnosed annually (Australian Institute of Health and Welfare (AIHW) 2011). Survival rates are also low, with about 30% of all deaths in Australia attributed to heart failure (Palazzuoli et al., 2007). The major risk factors for heart failure include age, lack of physical activity, poor eating habits leading to obesity, smoking and excessive alcohol intake (Palazzuoli et al., 2007). With many countries experiencing aging populations, heart failure is expected to become an even more prevalent problem (Marian and Nambi, 2004).

There is currently no standard for heart failure diagnosis, due to the complexity of the disease. In particular, there is no simple diagnostic test for heart failure. Early changes in the structure or function of the heart such as the compensation mechanisms mentioned above, can be detected using medical imaging technology, however, it is not practical or cost-effective to be performing imaging on all potential heart failure patients.

There are a number of non-invasive risk scoring systems which were designed to assess an individual's chances of developing cardiovascular disease, such as coronary heart disease, heart failure, cardiomyopathy, congenital heart disease, peripheral vascular disease and stroke. For example, the Framingham Risk Score is an algorithm for estimating the risk over 10 years of developing coronary heart disease, peripheral artery disease and heart failure (McKee et al., 1971). Other examples are the Boston Criteria for diagnosing heart failure (Carlson et al., 1985), which has been shown to have the highest sensitivity and specificity (Shamsham and Mitchell, 2000) and the Duke Criteria (Harlan et al., 1977). These types of criteria use a combination of patient medical history, physical examinations, routine clinical procedures and laboratory tests to reach a diagnostic conclusion (Krum et al., 2006) and are particularly useful in diagnosing advanced or severe heart failure. However, preventing progress of heart failure and clinical deterioration requires early diagnosis. An improvement in accuracy of non-invasive diagnosis of the early stages of heart failure is therefore required.

It will be clearly understood that, if a prior art publication is referred to herein, this reference does not constitute an admission that the publication forms part of the common general knowledge in the art in Australia or in any other country.

### SUMMARY OF INVENTION

The present invention is broadly directed to methods for the diagnosis of early stages of heart failure, in particular, classes I and II according to the NYHA classification. In particular, the invention relates to the identification and use of biomarkers with high correlation to early stage heart failure.

In a first aspect, the present invention provides a method for detecting early stage heart failure in a subject, the method comprising analysing a biological sample obtained from the subject and determining the concentration of at least one biomarker in the sample and assigning a heart failure classification to the subject if the concentration of the at least one biomarker is either higher or lower than a predefined reference concentration of the at least one biomarker. The predefined reference concentration of the at least one biomarker can be determined from a biological sample taken from a healthy subject.

In a second aspect, the invention provides a method of detecting early stage heart failure in a subject, the method comprising analysing a biological sample obtained from the subject and determining the concentration of at least one biomarker in the sample, determining the concentration of the at least one biomarker in a biological sample obtained from a healthy subject, and assigning a heart failure classification to the subject if the concentration of the at least one biomarker in the sample from the subject is either higher or lower than the concentration of the at least one biomarker in the biological sample obtained from the healthy subject.

In a third aspect, the invention provides a method for detecting early stage heart failure in a subject, the method comprising analysing a biological sample obtained from the subject and determining the concentration of at least one biomarker in the sample, wherein the at least one biomarker is selected from the group consisting of KLK1, TCPD, S10A7, DLDH, IGHA2, CAMP, KV110, NAMPT, COPB, SPR2A and HV311, and assigning a heart failure classification to the subject if the concentration of the at least one biomarker is either higher or lower than a predefined reference concentration of the at least one biomarker.

In a fourth aspect, the invention provides a method of detecting early stage heart failure in a subject, the method comprising analysing a biological sample obtained from the subject and determining the concentration of at least one biomarker in the sample, wherein the at least one biomarker is selected from the group consisting of KLK1, TCPD, S10A7, DLDH, IGHA2, CAMP, KV110, NAMPT, COPB, SPR2A and HV311, determining the concentration of the at least one biomarker in a biological sample obtained from a healthy subject, and assigning a heart failure classification to the subject if the concentration of the at least one biomarker in the sample from the subject is either higher or lower than the concentration of the at least one biomarker in the biological sample obtained from the healthy subject.

In a fifth aspect, the invention provides a method of screening for early stage heart failure in a subject, the method comprising analysing a biological sample obtained from the subject and determining the concentration of at least one biomarker in the sample and assigning a heart failure classification to the subject if the concentration of the at least one biomarker is either higher or lower than a predefined reference concentration of the at least one biomarker.

In a sixth aspect, the invention provides a kit for detecting the presence of at least one biomarker associated with early stage heart failure, the kit comprising a solid support having immobilized thereon at least one molecule that specifically binds to the at least one biomarker.

In a seventh aspect, the invention provides a kit for detecting the presence of at least one biomarker associated with early stage heart failure, wherein the at least one biomarker is selected from the group consisting of KLK1, TCPD, S10A7, DLDH, IGHA2, CAMP, KV110, NAMPT, COPB, SPR2A and HV311, the kit comprising a solid support having immobilized thereon at least one molecule that specifically binds to the at least one biomarker.

Throughout this specification, unless the context requires otherwise, the words “comprise”, “comprises” and “comprising” will be understood to imply the inclusion of a stated integer or group of integers but not the exclusion of any other integer or group of integers.

Any of the features described herein can be combined in any combination with any one or more of the other features described herein within the scope of the invention.

### BRIEF DESCRIPTION OF DRAWINGS

FIG. 1 is a graph showing the abundance of peptides from each protein identified by ProteinPilot database searches (Table 3) as determined from extracted ion chromatograms of LC-ESI-MS/MS data.

FIG. 2 is a series of graphs comparing the relative abundance of various salivary proteins in healthy controls and heart failure patients in NYHA Class I and Class III/IV, as determined by SWATH-MS. FIG. 2A, individual proteins validated by SWATH-MS; FIG. 2B, SPLC2 (BNP:Control); FIG. 2C, KLK1 (BNP:Control); FIG. 2D, KLK1:SPLC2 (BNP:Control); FIG. 2E, S10A7 (BNP:Control); FIG. 2F, S10A7:SPLC2 (BNP:Control); FIG. 2G, AACT (BNP:Control); and FIG. 2H, AACT:SPLC2 (BNP:Control).

FIG. 3 is a series of dot plots comparing the ratio of select salivary proteins in healthy controls and heart failure patients. FIG. 3A, KLK1:SPLC2; FIG. 3B, S10A7:SPLC2; and FIG. 3C, AACT:SPLC2.

FIGS. 4A, 4B and 4C are ROC curves for the salivary protein ratios in FIG. 3. FIG. 4A, KLK1:SPLC2; FIG. 4B, S10A7:SPCL2; and FIG. 4C, AACT:SPLC2.

FIG. 5 is a series of graphs comparing the relative abundance of various salivary proteins (KV110, NAMPT, COPB, SPR2A and HV311) in healthy controls and heart failure patients in NYHA Class I and Class III/IV, as determined by SWATH-MS.

FIG. 6 is an overlay of ROC curves for comparisons of the combination of salivary proteins shown in FIG. 5 between various cohorts (NYHA Class I, NYHA Class III/IV and controls).

FIG. 7 is a series of graphs comparing the relative abundance of various salivary proteins (KLK1, TCPD, S10A7, DLDH, IGHA2 and CAMP) in healthy controls and heart failure patients in NYHA Class I and Class III/IV, as determined by SWATH-MS.

FIG. 8 is an overlay of ROC curves for comparisons of the combination of salivary proteins shown in FIG. 7 between various cohorts (NYHA Class I, NYHA Class III/IV and controls).

FIG. 9 is a series of graphs comparing the concentration of various salivary proteins (S10A7, KLK1 and CAMP) in healthy controls, individuals with high risk of developing heart failure and heart failure patients, as determined by immunoassays; and ROC curves for comparisons of the combination salivary proteins. A prediction score was generated by combining the concentration of these salivary proteins. FIG. 9A, S10A7; FIG. 9B, CAMP; FIG. 9C, KLK1; FIG. 9D, combined prediction score of the salivary proteins; FIG. 9E, ROC curve for comparison of the combination of salivary proteins between heart failure patients and controls; FIG. 9F, ROC curve for comparison of the combination of salivary proteins between SCREEN-HF cohorts and controls.

FIG. 10 is a graph showing the prediction score between study subjects who have developed cardiovascular disease after enrolment in the study, and those who have no cardiovascular disease-related hospital admission.

FIG. 11 (A) Western blotting of KLK1, TCPD, S10A7, DLDH, IGHA2 and CAMP in saliva samples of 6 healthy controls and 6 heart failure patients. (B) Average relative band intensity with standard error of KLK1, TCPD, S10A7, DLDH, IGHA2 and CAMP in saliva samples of healthy control and heart failure patients.

FIG. 12 is a Western blot of S10A7 in additional saliva samples of 12 healthy controls and 12 heart failure patients.

### DESCRIPTION OF EMBODIMENTS

### Abbreviations

The following abbreviations are used throughout:
AACT=alpha 1 anti-chymotrypsin
BNP=brain natriuretic peptide
CAMP=Cathelicidin antimicrobial peptide
COPB=coatomer subunit beta
DLDH=Dihydrolipoyl dehydrogenase, mitochondrial
ESI=electron spray ionization
GELS=gelsolin
h=hour
HV311=Ig heavy chain V-III region KOL
IGHA2=Ig alpha-2 chain C region
IGJ=immunoglobulin J chain
IQR=interquartile range
KLK1=kallikrein 1
KV110=Ig kappa chain V-I region HK102
LC=liquid chromatography
LC-ESI-MS/MS=liquid chromatograph-electrospray ionization-tandem mass spectrometry
LPLC1=long palate lung and nasal epithelium carcinoma-associated protein 1
min=minute(s)
MMP9=matrix metalloproteinase-9
MS=mass spectrometry
MS/MS=tandem mass spectrometry
NAMPT=nicotinamide phosphoribosyltransferase
NPV=negative predictive value
NYHA=New York Heart Association
PBS=phosphate buffered saline
PPV=positive predictive value
rcf=relative centrifugal force
ROC=receiver operating characteristic
s=second(s)
S10A7=S100 calcium binding protein A7
SPLC2=short palate lung and nasal associated protein 2
SPR2A=small proline-rich protein 2A
SWATH=sequential window acquisition of all theoretical fragment ion spectra
TCPD=T-complex protein 1 subunit delta
TOF=time of flight
VIME=vimentin

The present invention is predicated in part on the discovery that proteins in a biological sample taken from a subject with early stage heart failure are differentially abundant when compared to a biological sample taken from a healthy subject. The present inventors have used high abundant protein depletion and SWATH-MS to identify salivary proteins as putative biomarkers having diagnostic utility in the early stages of heart failure.

Accordingly, in a first aspect, the invention provides a method for detecting early stage heart failure in a subject, the method comprising analysing a biological sample obtained from the subject and determining the concentration of at least one biomarker in the sample and assigning a heart failure classification to the subject if the concentration of the at least one biomarker is either higher or lower than a predefined reference concentration of the at least one biomarker. The predefined reference concentration of the at least one biomarker can be determined from a biological sample taken from a healthy subject.

For the purposes of this invention, the phrase “early stage(s)” to describe a stage of heart failure, refers to the functional classifications NYHA Class I and/or NYHA Class II, as defined by the New York Heart Association.

The term “biological sample” is used herein to refer to a sample that is extracted from a subject. The term encompasses untreated, treated, diluted or concentrated biological samples. The biological sample obtained from the subject can be any suitable sample, such as whole blood, serum or plasma. Preferably, the biological sample is obtained from the buccal cavity of the subject. The biological sample can therefore be sputum or saliva. In accordance with the invention providing a non-invasive, cost-effective method for diagnosing early stage heart failure, the biological sample obtained from the subject is preferably saliva.

The at least one biomarker is a protein present in the biological sample that has been identified as having a correlation with early stage heart failure. The biological sample can be analysed for the concentration of at least one, two, three, four, five, six, etc., biomarkers. For example, the at least one biomarker can be any number of proteins selected from the group consisting of KLK1, TCPD, S10A7, DLDH, IGHA2, CAMP, KV110, NAMPT, COPB, SPR2A and HV311. In one embodiment, the at least one biomarker is selected from the group of proteins consisting of KLK1, TCPD, S10A7, DLDH, IGHA2 and CAMP. Preferably, the at least one biomarker is a biomarker panel consisting of two, three, four, five, or all six of these proteins. In a particularly preferred embodiment, the biomarker panel comprises KLK1, S10A7, and CAMP. In an alternative embodiment, the at least one biomarker is selected from the group consisting of KV110, NAMPT, COPB, SPR2A and HV311. In a particularly preferred embodiment, the at least one biomarker is a biomarker panel consisting of two, three, four or all five of these proteins.

The predefined reference concentration for a biomarker can be in the form of a range of concentrations, such that a concentration of a biomarker outside the range is indicative of an early stage of heart failure. Alternatively, the predefined reference concentration for a biomarker can be in the form of a particular value, such that a concentration of a biomarker either higher or lower than the value is indicative of an early stage of heart failure. Therefore, for each biomarker used in the detection of early stage heart failure in a subject, a predefined reference concentration of the biomarker in a biological sample from a healthy subject has been determined, or is known.

In the context of this invention and with respect to determining predefined reference concentrations of the at least one biomarker from a biological sample taken from a healthy subject, a “healthy subject” is a subject that does not have heart failure. That is, a healthy subject is a subject that is not suffering any outward symptoms of heart failure, and would not be classified in NYHA Class I or Class II.

The present inventors have surprisingly found that particular proteins have increased abundance in saliva from subjects classified in NYHA Class I or Class II when compared to the abundance of the same protein in a healthy subject. Conversely, particular proteins have decreased abundance in saliva from subjects classified in NYHA Class I or Class II when compared to the abundance of the same protein in a healthy subject.

Although a heart failure classification can be assigned to a subject based on the concentration of just one biomarker in a biological sample from the subject, it is advantageous to base the assignation of classification on the concentration of two, three, four, five or more biomarkers in the biological sample, as a higher degree of certainty of classification could be achieved by using more biomarkers.

When using a biomarker panel consisting of two or more biomarkers to detect early stage heart failure in a subject, the panel can consist of biomarkers that have a higher concentration in saliva from a heart failure subject than for the same biomarkers in saliva from a healthy subject. Alternatively, the panel can consist of biomarkers that have a lower concentration in saliva from a heart failure subject than from the same biomarkers in saliva from a healthy subject. In a further alternative, the panel can consist of a combination of biomarkers, wherein at least one biomarker has a higher concentration in saliva from a heart failure subject than for the same biomarker in saliva from a healthy subject and at least one biomarker has a lower concentration in saliva from a heart failure subject than for the same biomarker in saliva from a healthy subject.

In a second aspect, the invention provides a method of detecting early stage heart failure in a subject, the method comprising analysing a biological sample obtained from the subject and determining the concentration of at least one biomarker in the sample, determining the concentration of the at least one biomarker in a biological sample obtained from a healthy subject, and assigning a heart failure classification to the subject if the concentration of the at least one biomarker in the sample from the subject is either higher or lower than the concentration of the at least one biomarker in the biological sample obtained from the healthy subject.

The concentration of the at least one biomarker in a biological sample, whether from a potential heart failure subject or a healthy subject, can be determined by any suitable means for determining protein concentration. For example, the concentration can be determined by mass spectrometry analysis. Comparison of peak intensity for a particular biomarker in the mass spectrum of a sample from a potential heart failure subject and the mass spectrum of a sample from a healthy subject can provide an indication of the relative difference in abundance of the biomarker in the two samples. A more accurate comparison can be obtained using SWATH-MS as detailed in the Examples, below.

Alternatively, determining the concentration of a least one biomarker in a biological sample can be undertaken using a reagent or reagents that specifically bind to the at least one biomarker. For example, the reagent could comprise an antibody to an epitope of the biomarker, with the antibody optionally including a label (e.g. a fluorescent tag) for detecting the presence of the antibody-biomarker complex.

In a third aspect, the invention provides a method for detecting early stage heart failure in a subject, the method comprising analysing a biological sample obtained from the subject and determining the concentration of at least one biomarker in the sample, wherein the at least one biomarker is selected from the group of proteins consisting of KLK1, TCPD, S10A7, DLDH, IGHA2, CAMP, KV110, NAMPT, COPB, SPR2A and HV311, and assigning a heart failure classification to the subject if the concentration of the at least one biomarker is higher or lower than a predefined reference concentration of the at least one biomarker. The predefined reference concentration of the at least one biomarker can be determined from a biological sample taken from a healthy subject.

The biological sample can be analysed for the concentration of at least one, two, three, four, five, six, seven, eight, nine, ten, or all eleven of the proteins. Although a heart failure classification can be assigned to a subject based on the concentration of just one protein from the biological sample, it is advantageous to base the assignation of classification on the concentration of two, three, four, five, six, seven, eight, nine, ten, or eleven proteins in the biological sample, as a higher degree of certainty of classification could be achieved by using more biomarkers.

The certainty of classification can be assessed by determining the sensitivity and specificity of the comparative data.

In a fourth aspect, the invention provides a method of detecting early stage heart failure in a subject, the method comprising analysing a biological sample obtained from the subject and determining the concentration of at least one biomarker in the sample, wherein the at least one biomarker is selected from the group of proteins consisting of KLK1, TCPD, S10A7, DLDH, IGHA2, CAMP, KV110, NAMPT, COPB, SPR2A and HV311, determining the concentration of the at least one biomarker in a biological sample obtained from a healthy subject, and assigning a heart failure classification to the subject if the concentration of the at least one biomarker in the sample from the subject is higher or lower than the concentration of the at least one biomarker in the biological sample obtained from the healthy subject.

In a fifth aspect, the invention provides a kit for detecting the presence of at least one biomarker associated with early stage heart failure, the kit comprising a solid support having immobilized thereon at least one molecule that specifically binds to the at least one biomarker.

The at least one molecule that specifically binds to the at least one biomarker can be any suitable molecule. Preferably, the at least one molecule comprises an antibody that specifically binds to the at least one biomarker. The solid support can therefore have one, two, three, four, etc. antibodies immobilized thereon.

The solid support can be any suitable material that can be modified as appropriate for the immobilization of antibodies and is amenable to at least one detection method. Representative examples of materials suitable for the solid support include glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses and plastics. The solid support can allow for optical detection without appreciably fluorescing.

The solid support can be planar, although other configurations of substrates can be utilized. For example, the solid support could be a tube with antibodies placed on the inside surface.

In a sixth aspect, the invention provides a kit for detecting the presence of at least one biomarker associated with early stage heart failure, wherein the at least one biomarker is selected from the group consisting of KLK1, TCPD, S10A7, DLDH, IGHA2, CAMP, KV110, NAMPT, COPB, SPR2A and HV311, the kit comprising a solid support having immobilized thereon at least one molecule that specifically binds to the at least one biomarker.

### EXAMPLES

### Example 1

### Materials and Methods

### Study Participants

This study was approved by the University of Queensland Medical Ethical Institutional Board and Mater Health Services Human Research Ethics Committee and by the Royal Brisbane and Women's Hospital Research Governance. All study participants were >18 years of age and gave informed consent before sample collection. The exclusion criteria for the healthy controls were based on a simple questionnaire asking volunteers to indicate the existence of any comorbid diseases and oral diseases (e.g. periodontal disease and gingivitis, autoimmune, infectious, musculoskeletal, or malignant disease, and recent operation or trauma). If any condition existed, the participants were excluded from the study. The volunteers were from Caucasian and Asian ethnic origins, had no symptoms of fever or cold, and had good oral hygiene.

A total of 30 healthy controls and 33 symptomatic heart failure patients were recruited from the University of Queensland, the Mater Adult Hospital or the Royal Brisbane and Women's Hospital in Brisbane, Australia from January 2012 to July 2014. Patients were classified using New York Heart Association (NYHA) functional classification system by cardiologists at Mater Adult Hospital and Royal Brisbane and Women's Hospital based on their clinical symptoms. All patients participating in the study were classified as NYHA class III or IV patients. The mean age of heart failure patients was 67.6 and the mean age of healthy controls was 49.7. Males comprised 63.3% of the heart failure patient cohort and 43.3% of the healthy control cohort.

### Saliva Sample Collection

Whole mouth unstimulated resting saliva was collected from early and late stage heart failure patients and from healthy controls according to previously published methods (Martinet W et al., 2003; Punyadeera C et al., 2011; Foo J Y et al., 2013; Castagnola M et al., 2011; Helmerhorst E J and Oppenheim F G, 2007; Loo J A et al., 2010). Volunteers were asked to refrain from eating or drinking (except for water) for at least 30 minutes prior to saliva collection. Volunteers were asked to rinse their mouth with water to remove food particles and debris, to tilt their head forward and down, pool saliva in their mouth and expectorate into Falcon tubes (50 mL, Greiner, Germany) on ice. Samples were transferred to the laboratory on dry ice and aliquoted into protein lo-bind Eppendorf tubes (Eppendorf, USA) and stored at −80° C. for later analysis.

### Total Protein Concentrations in Saliva Samples

For initial screening, total protein concentrations in saliva samples from patients (n=10) and controls (n=10) were measured using a 2D Quant kit (GE Healthcare Bio-Sciences AB, Sweden). The absorbance was measured at 480 nm using a SpectraMax® 190 plate reader (Molecular Devices, LLC, California, USA). Quick Start™ Bradford Protein Assay (Bio-Rad, USA) was used to quantify the total protein concentrations in saliva samples from patients (n=30) and controls (n=30) for the SWATH-MS validation (see below).

### Saliva Sample Preparation for Mass Spectrometry Analysis

Saliva samples normalized for protein content collected from heart failure patients and healthy controls were separately pooled. Equal amounts of total protein from each individual were pooled to give 10 mg of total pooled protein each for controls and patients. Pooled samples were processed with a ProteoMiner® small capacity kit (Bio-Rad, Hercules, Calif.) according to the manufacturer's instructions. Bead packed bed (20 uL) was added to pooled saliva and incubated at 25° C. for 16 hours on a rotational shaker. Beads were pelleted by centrifugation at 1,000 relative centrifugal force (rcf) for 1 minute and the supernatant discarded. Beads were washed three times with phosphate buffered saline (PBS) and bound proteins were eluted in 8 M urea, 2% CHAPS and 5% acetic acid (20 μL). Eluted proteins were precipitated by the addition of 1:1 methanol:acetone (80 μL), incubation at −20° C. for 16 hours, and centrifugation at 18,000 rcf for 10 minutes. The protein pellets were resuspended in 50 mM Tris-HCl buffer pH 8 with 1% SDS. Cysteines were reduced by addition of DTT to 10 mM and incubation at 95° C. for 10 min, and then alkylated by addition of acrylamide to 25 mM and incubation at 23° C. for 1 h. Proteins were precipitated as above, resuspended in 50 mM NH₄HCO₃ (50 μL) with proteomics grade trypsin (1 μg) (SigmaAlrdich, USA) and incubated at 37° C. for 16 h.

For SWATH-MS validation using individual samples, saliva containing 50 μg of total protein was supplemented with an equal volume of 100 mM Tris-HCl buffer pH 8, 2% SDS and 20 mM DTT and incubated at 95° C. for 10 min. Proteins were then alkylated, precipitated and digested as above.

### Mass Spectrometry and Data Analysis

Peptides were desalted using C18 Zip Tips (Millipore, USA) and analyzed by LC-ESI-MS/MS using a Prominence nanoLC system (Shimadzu, Japan) on a Triple TOF 5600 mass spectrometer with a Nanospray III interface (AB SCIEX) essentially as previously described (Foo et al., 2013; Ovchinnikov et al., 2012). Approximately 2 μg of peptides were desalted on an Agilent C18 trap (300 Å pore size, 5 μm particle size, 0.3 mm i.d.×5 mm) at a flow rate of 30 μL/min for 3 min, and then separated on a Vydac EVEREST reversed-phase C18 HPLC column (300 Å pore size, 5 μm particle size, 150 μm i.d.×150 mm) at a flow rate of 1 μL/min. Peptides were separated with a gradient of 1-10% buffer B over 2 min followed by 10-60% buffer B over 45 min, with buffer A (1% acetonitrile and 0.1% formic acid) and buffer B (80% acetonitrile with 0.1% formic acid). Gas and voltage settings were adjusted as required. An MS-TOF scan from an m/z of 350-1800 was performed for 0.5 s followed by information dependent acquisition of MS/MS with automated CE selection of the top 20 peptides from m/z of 40-1800 for 0.05 s per spectrum. Identical LC parameters were used for SWATH analyses, with an MS-TOF scan from an m/z of 350-1800 for 0.05 s followed by high sensitivity information independent acquisition with 26 m/z isolation windows with 1 m/z window overlap each for 0.1 s across an m/z range of 400-1250. Collision energy was automatically assigned by the Analyst software (AB SCIEX) based on m/z window ranges.

Proteins were identified using ProteinPilot (AB SCIEX), searching the LudwigNR database (downloaded from http://apcf.edu.au as at 27 Jan. 2012; 16,818,973 sequences; 5,891,363,821 residues) using standard settings: Sample type, identification; Cysteine alkylation, none; Instrument, Triple-TOF 5600; Species, no restriction; ID focus, biological modifications; Enzyme, trypsin; Search effort, thorough ID. False discovery rate analysis using ProteinPilot was performed on all searches. Peptides identified with greater than 99% confidence and with a local false discovery rate of less than 1% were included for further analysis. Semi-quantitative comparison of protein abundance based on protein rank, score, percent peptide coverage and number of peptides was performed as previously described (Bailey and Schulz, 2013). Extracted ion chromatograms were obtained using Peak View 1.1. The ProteinPilot data were used as ion libraries for SWATH analyses. Protein abundance was measured automatically with Peak View 1.2 Software with standard settings. The abundance of each protein was normalized to the total abundance of identified proteins in each individual sample, log-transformed and compared using ANOVA. Data generated with SWATH analyses were analysed for protein significance using an open-sourced statistical package MS stats (Clough et al., 2012; Chang et al., 2012) based on R (R Development Core Team, 2011). Group comparison function was used to compare significant changes in protein abundance between heart failure patients and controls.

### Example 2

### Identification of Proteins Via LC-ESI-MS/MS

Putative novel salivary protein biomarkers for heart failure were identified by separately pooling saliva from patients with elevated BNP and healthy controls, performing ProteoMiner dynamic range reduction, digesting proteins with trypsin and identifying peptides using LC-ESI-MS/MS and database searching. To detect proteins with altered abundance between heart failure patients and controls, a semi-quantitative approach was used to compare the rank, score, precent peptide coverage and number of peptides identified for each protein. This semi-quantitative approach identified multiple putative differentially abundant proteins as presented in Table 2.

TABLE 2
Differentially abundant salivary proteins, comparing heart failure patients to controls

| | N | | | Score | | | % Cov | | | Peptides(95%) | | |
|---|---|---|---|---|---|---|---|---|---|---|---|---|
| Protein Accession | B | C | ² | B | C | ² | B | C | ² | B | C | ² |
| sp\|Q96DR5\|SPLC2_HUMAN | 53 | 16 | 37 | 4 | 18.51 | −14.51 | 8.83 | 40.96 | −32.12 | 2 | 9 | −7 |
| sp\|P22079\|PERL_HUMAN | 87* | 27 | 60 | | 12 | −12 | | 13.06 | −13.06 | | 6 | −6 |
| sp\|Q08380\|LG3BP_HUMAN | 72 | 25 | 47 | 2 | 12.02 | −10.02 | 2.22 | 18.12 | −15.90 | 1 | 6 | −5 |
| sp\|P06396\|GELS_HUMAN | 15 | 7 | 8 | 13.44 | 22.07 | −8.63 | 18.16 | 27.62 | −9.46 | 7 | 12 | −5 |
| sp\|P08670\|VIME_HUMAN | 79 | 51 | 28 | 2 | 8 | −6 | 2.15 | 9.23 | −7.08 | 1 | 4 | −3 |
| sp\|P07237\|PDIA1_HUMAN | 61 | 38 | 23 | 4 | 10 | −6 | 3.54 | 10.24 | −6.70 | 2 | 5 | −3 |
| sp\|P07737\|PROF1_HUMAN | 21 | 30 | −9 | 12 | 11.62 | 0.38 | 55.71 | 55.71 | 0.00 | 7 | 7 | 0 |
| sp\|P01833\|PIGR_HUMAN | 18 | 33 | −15 | 12 | 10.59 | 1.41 | 12.30 | 8.51 | 3.79 | 6 | 5 | 1 |
| sp\|P04075\|ALDOA_HUMAN | 22 | 52 | −30 | 10 | 8 | 2 | 25.27 | 18.96 | 6.31 | 5 | 4 | 1 |
| sp\|P06870\|KLK1_HUMAN | 47 | 101 | −54 | 4.09 | 2 | 2.09 | 14.12 | 9.16 | 4.96 | 3 | 1 | 2 |
| sp\|P0CG06\|LAC3_HUMAN | 31 | 74 | −43 | 8 | 4 | 4 | 46.23 | 32.08 | 14.15 | 4 | 2 | 2 |
| sP\|P01591\|IGJ_HUMAN | 45 | 128* | −83 | 5.54 | | 5.54 | 23.27 | | 23.27 | 4 | | 4 |
| sP\|P14780\|MMP9_HUMAN | 42 | 128* | −86 | 6 | | 6 | 5.94 | | 5.94 | 3 | | 3 |
| sp\|Q8TDL5\|LPLC1_HUMAN | 10 | 60 | −50 | 17.07 | 6 | 11.07 | 22.73 | 8.88 | 13.85 | 11 | 3 | 8 |

B, BNP; C, Control; ², BNP - Control; N, protein rank; *not identified, lowest rank.

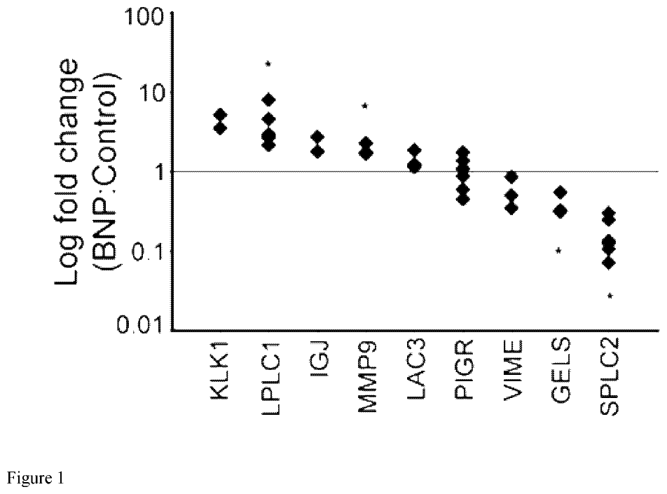

For initial validation of these putative biomarkers, the abundance of peptides from each protein identified by ProteinPilot database searches (Table 3) as determined from extracted ion chromatograms of LC-ESI-MS/MS data (FIG. 1) was compared. Comparison of peptide abundances identified two proteins with significantly higher abundance (long palate, lung and nasal epithelium carcinoma-associated protein 1, LPLC1 (P=0.0004) and matrix metalloproteinase-9, MMP9 (P=0.02)) and two with significantly lower abundance (gelsolin, GELS (P=0.03) and short palate lung and nasal associated protein 2, SPLC2 (P=0.0003)) in heart failure patients compared with the control samples. Several additional proteins showed large changes in abundance (kallikrein 1, KLK1; immunoglobulin J chain, IGJ; and vimentin, VIME) which could not be statistically compared due to the small number of confidently identified peptides detected. This initial analysis therefore identified several putative salivary protein biomarkers of heart failure.

TABLE 3
Relative abundances of peptides for each protein identified
using ProteinPilot

| Protein Accession | Peptide | ZMass | m/z | z | Score |
|---|---|---|---|---|---|
| sp\|P01591\|IGJ_HUMAN | CYTAVVPLVYGGETK | 0.0008 | 835.92 | 2 | 16 |
| sp\|P01591\|IGJ_HUMAN | IIVPLNNR | −0.0028 | 469.78 | 2 | 8 |
| sp\|P01591\|IGJ_HUMAN | MVETALTPDACYPD | 0.0015 | 798.84 | 2 | 10 |
| sp\|P01833\|PIGR_HUMAN | CPLLVDSEGWVK | −0.0043 | 708.85 | 2 | 10 |
| sp\|P01833\|PIGR_HUMAN | DGSFSVVITGLR | −0.0022 | 625.83 | 2 | 15 |
| sp\|P01833\|PIGR_HUMAN | ILLNPQDK | −0.0031 | 470.77 | 2 | 8 |
| sp\|P01833\|PIGR_HUMAN | LVSLTLNLVTR | −0.0015 | 614.88 | 2 | 16 |
| sp\|P01833\|PIGR_HUMAN | NADLQVLICPEPELVYEDLR | 0.0104 | 747.73 | 3 | 18 |
| sp\|P01833\|PIGR_HUMAN | VYTVDLGR | −0.0021 | 461.74 | 2 | 7 |
| sp\|P06396\|GELS_HUMAN | AQPVQVAEGSEPDGFWEALGGK | −0.0036 | 1136.54 | 2 | 16 |
| sp\|P06396\|GELS_HUMAN | EPAHLMSLFGGKPMITYK | 0.0006 | 508.77 | 4 | 10 |
| sp\|P06396\|GELS_HUMAN | EVQGFESATFLGYFK | 0.0017 | 861.92 | 2 | 9 |
| sp\|P06396\|GELS_HUMAN | HVVPNEVVVQR | 0.0011 | 638.36 | 2 | 10 |
| sp\|P06870\|KLK1_HUMAN | LTEPADTITDAVK | −0.0024 | 687.35 | 2 | 12 |
| sp\|P06870\|KLK1_HUMAN | QADEDYSHDLMLLR | −0.0019 | 853.39 | 2 | 12 |
| sp\|P08670\|VIME_HUMAN | EEAENTLQSFR | −0.0073 | 662.30 | 2 | 11 |
| sp\|P08670\|VIME_HUMAN | EYQDLLNVK | −0.001 | 561.29 | 2 | 10 |
| sp\|P08670\|VIME_HUMAN | ILLAELEQLK | −0.0036 | 585.35 | 2 | 8 |
| sp\|P0CG06\|LAC3_HUMAN | AAPSVTLFPPSSEELQANK | 0.0026 | 662.67 | 3 | 16 |
| sp\|P0CG06\|LAC3_HUMAN | AAPSVTLFPPSSEELQANK | 0.0024 | 993.51 | 2 | 16 |
| sp\|P0CG06\|LAC3_HUMAN | SYSCQVTHEGSTVEK | −0.0038 | 575.92 | 3 | 12 |
| sp\|P0CG06\|LAC3_HUMAN | YAASSYLSLTPEQWK | 0.0013 | 872.43 | 2 | 16 |
| sp\|P0CG06\|LAC3_HUMAN | YAASSYLSLTPEQWK | 0.0031 | 581.95 | 3 | 17 |
| sp\|P14780\|MMP9_HUMAN | LGLGADVAQVTGALR | −0.0032 | 720.90 | 2 | 9 |
| sp\|P14780\|MMP9_HUMAN | QLSLPETGELDSATLK | 0.0004 | 851.44 | 2 | 11 |
| sp\|P14780\|MMP9_HUMAN | SLGPALLLLQK | −0.0047 | 576.86 | 2 | 11 |
| sp\|Q8TDL5\|LPLC1_HUMAN | ALGFEAAESSLTK | −0.0029 | 662.33 | 2 | 19 |
| sp\|Q8TDL5\|LPLC1_HUMAN | DALVLTPASLWKPSSPVSQ | −0.0008 | 998.53 | 2 | 15 |
| sp\|Q8TDL5\|LPLC1_HUMAN | GDQLILNLNNISSDR | −0.011 | 836.42 | 2 | 14 |
| sp\|Q8TDL5\|LPLC1_HUMAN | ILTQDTPEFFIDQGHAK | 0.0046 | 653.99 | 3 | 13 |
| sp\|Q8TDL5\|LPLC1_HUMAN | IPLDMVAGFNTPLVK | −0.0016 | 807.94 | 2 | 19 |
| sp\|Q8TDL5\|LPLC1_HUMAN | SGVPVSLVK | −0.0006 | 443.27 | 2 | 9 |
| sp\|Q8TDL5\|LPLC1_HUMAN | SSIGLINEK | −0.0023 | 480.76 | 2 | 10 |
| sp\|Q96DR5\|SPLC2_HUMAN | FVNSVINTLK | −0.0028 | 567.82 | 2 | 10 |
| sp\|Q96DR5\|SPLC2_HUMAN | ISNSLILDVK | −0.0023 | 551.32 | 2 | 14 |
| sp\|Q96DR5\|SPLC2_HUMAN | LEPVLHEGLETVDNTLK | 0.0002 | 636.34 | 3 | 13 |
| sp\|Q96DR5\|SPLC2_HUMAN | LLNNVISK | −0.0029 | 450.77 | 2 | 9 |
| sp\|Q96DR5\|SPLC2_HUMAN | LLPTNTDIFGLK | −0.0007 | 666.37 | 2 | 10 |
| sp\|Q96DR5\|SPLC2_HUMAN | VDLGVLQK | −0.0006 | 436.26 | 2 | 10 |

### Example 3

Validation with SWATH-MS

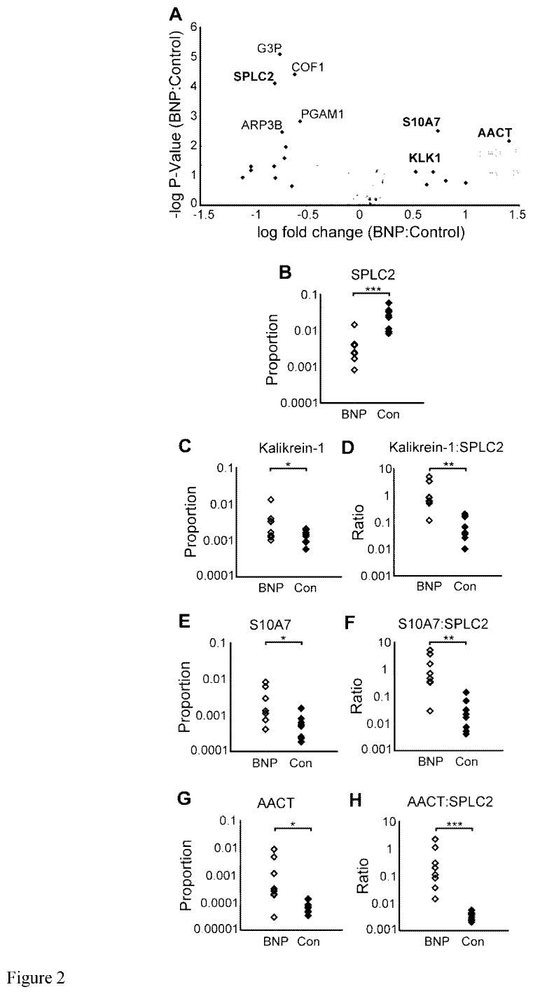

To validate the novel putative biomarkers identified from ProteoMiner® analysis of pooled samples, SWATH-MS detection was performed on individual saliva samples collected from heart failure patients and controls. Unbiased SWATH-MS proteomic comparison of saliva from heart failure patients and controls resulted in the identification of seven proteins with >2-fold difference in abundance and adjusted P<0.01. This included the SPLC2 protein identified by ProteoMiner analysis as a putative heart failure biomarker. The relative abundance of SPLC2 was 1.89-fold lower in heart failure patients than in controls. Saliva with high specificity (almost complete group separation) (see FIG. 2A, adjusted P<0.0001), validated SPLC2 as a salivary protein biomarker for heart failure. KLK1 was also putatively identified by ProteoMiner analysis as a potential biomarker due to its higher abundance in saliva from heart failure patients then in saliva from controls (FIG. 1). The increased abundance of KLK1 was also validated by SWATH-MS analysis, which showed a 1.3-fold increase in abundance in heart failure patients compared to controls (FIG. 2B, adjusted P=<0.0001).

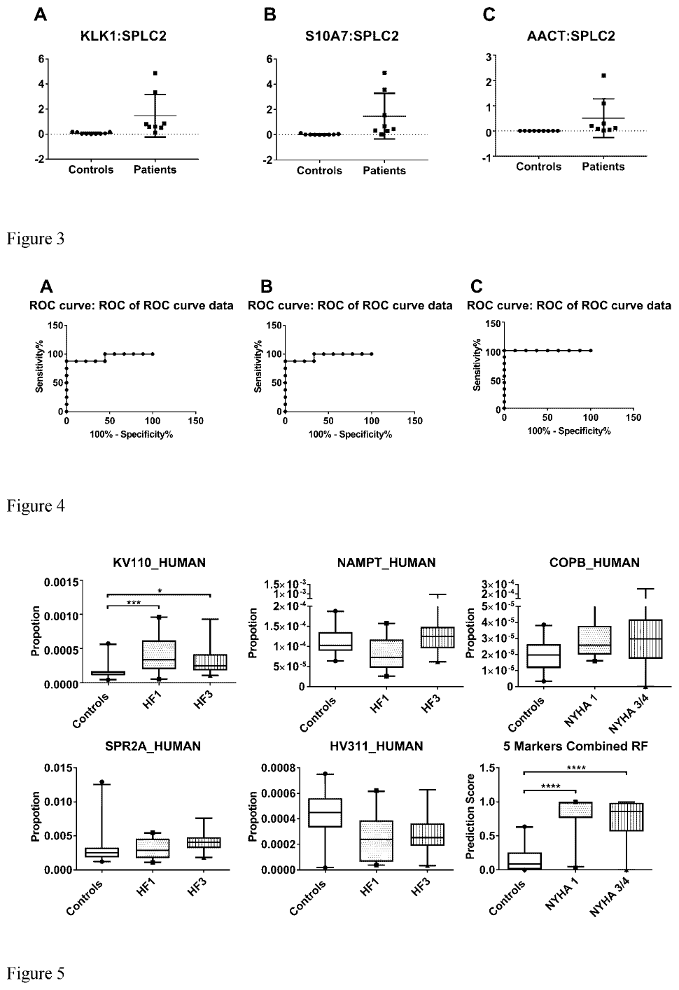

As SPLC2 abundance was decreased and KLK1 abundance increased in heart failure patients compared to controls, the utility of a ratio of the abundance of these individually validated biomarkers for identifying heart failure was investigated. A large and highly significant discrimination between heart failure patients and controls was observed, with a 5.3-fold difference in ratio and high specificity (FIG. 2C, P=0.00001). A Receiver Operating Characteristic (ROC) curve analysis was undertaken to determine the diagnostic power of SPLC2 and KLK1 as biomarkers. The analysis of KLK:SPLC2 (FIG. 3A, FIG. 4A) shows an area under the curve (AUC) value of 0.75 with a sensitivity of 70.0% and a specificity of 66.7%.

### Example 4

### Predictive Power of Biomarker Panel

The predictive power of a panel comprising the putative biomarkers KV110, NAMPT, COPB, SPR2A and HV311 (FIG. 5) for early stage heart failure was assessed using MSstats (Clough et al., 2012; Chang et al., 2012), which is based on R (R Development Core Team, 2011). The sensitivity and specificity of the combination of biomarkers in the various cohorts (NYHA Class I, n=20; NYHA Class III/IV, n=19; healthy controls, n=20) are set out in Table 4.

TABLE 4
Sensitivity and specificity of the combination of biomarkers

| | | | | Positive | Negative |
|---|---|---|---|---|---|
| | | | | Predictive | Predictive |
| | AUC | Sensitivity | Specificity | Value (PPV) | Value (NPV) |
| Class I vs | 0.96 | 95.0% | 90.0% | 94.7% | 90.5% |
| Controls | | | | | |
| Class III/IV | 0.85 | 79.0% | 95.0% | 82.6% | 93.8% |
| vs Controls | | | | | |
| Class III/IV | 0.65 | 73.8% | 60.0% | 70.6% | 63.7% |
| vs Class I | | | | | |

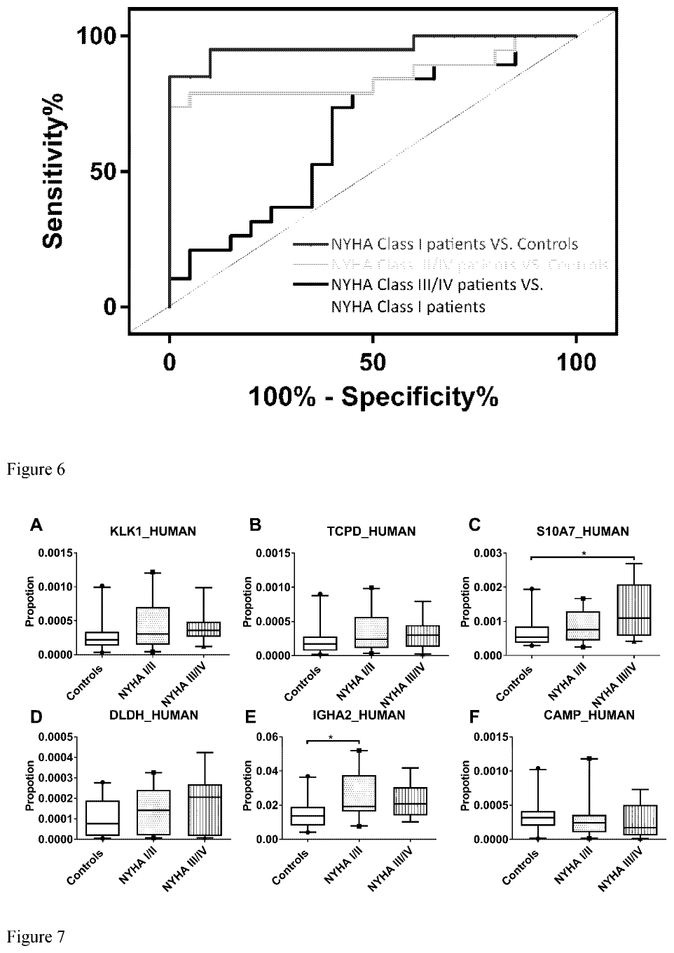

The ROC curves in FIG. 6 provide a useful summary of the diagnostic potential of the combination of five biomarkers, KV110, NAMPT, COPB, SPR2A and HV311. The closer the area under a ROC curve is to 1, the better the diagnostic potential. The ROC curve for the combination of five biomarkers in NYHA Class I patients compared to the five biomarkers in healthy controls has an AUC of 0.96, a sensitivity of 95.0% and a specificity of 90.0% (FIG. 6). These results are indicative of high diagnostic capability of the combination of five biomarkers.

The predictive power of a panel comprising the putative biomarkers KLK1, TCPD, S10A7, DLDH, IGHA2 and CAMP (FIG. 7) for early stage heart failure was assessed using MSstats (Clough et al., 2012; Chang et al., 2012), which is based on R (R Development Core Team, 2011). The sensitivity and specificity of the combination of biomarkers in the various cohorts (NYHA Class I, n=20; NYHA Class III/IV, n=19; healthy controls, n=20) are set out in Table 5.

TABLE 5
Sensitivity and specificity of the combination of biomarkers

| | AUC | Sensitivity | Specificity | PPV | NPV |
|---|---|---|---|---|---|
| Class I vs | | | | | |
| Controls | | | | | |
| Class III/IV | 0.91 | 84.2% | 85.0% | 85.0% | 84.2% |
| vs Controls | | | | | |
| Class III/IV | 0.71 | 68.4% | 70% | 70.0% | 68.5% |
| vs Class I | | | | | |

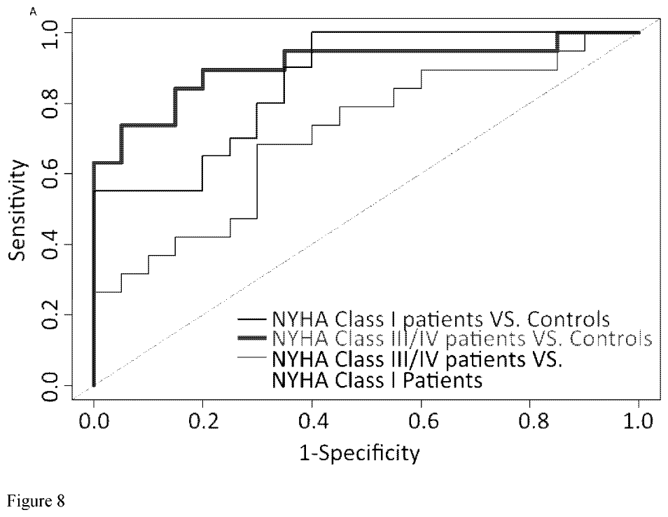

The ROC curves in FIG. 8 provide a useful summary of the diagnostic potential of the combination of six biomarkers, KLK1, TCPD, S10A7, DLDH, IGHA2 and CAMP. The closer the area under a ROC curve is to 1, the better the diagnostic potential. The ROC curve for the combination of six biomarkers in NYHA Class I patients compared to the six biomarkers in healthy controls has an AUC of 0.86, a sensitivity of 80.0% and a specificity of 70.0% (FIG. 8). These results are indicative of high diagnostic capability of the combination of six biomarkers.

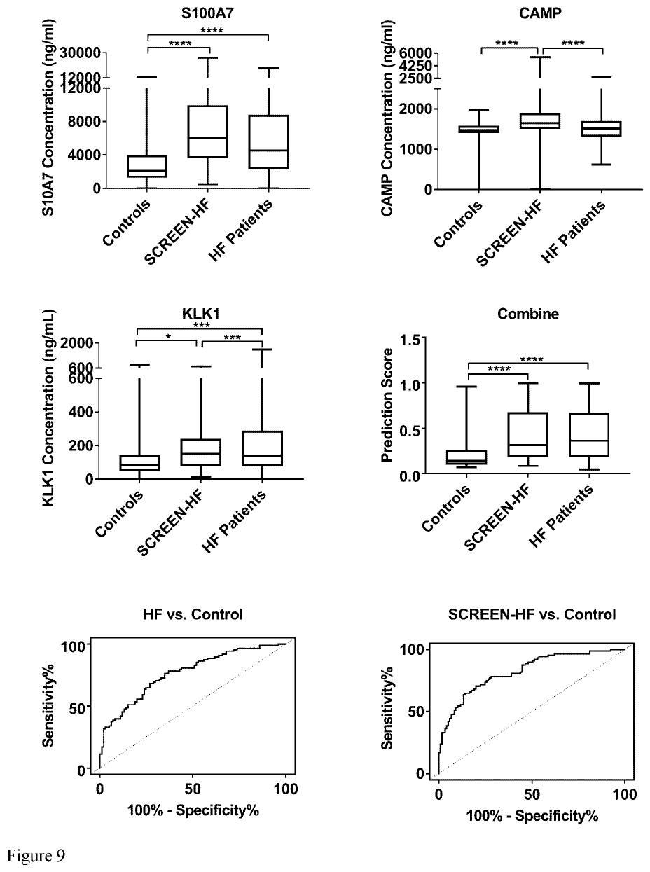

The predictive power of a panel comprising the putative biomarkers KLK1, S10A7 and CAMP (FIG. 9) for individuals with high risk of developing heart failure was assessed using MSstats (Clough et al., 2012; Chang et al., 2012), which is based on R (R Development Core Team, 2011). The sensitivity and specificity of the combination of biomarkers in the various cohorts (heart failure patient, n=100; individuals with high risk of developing heart failure (SCREEN-HF), n=121; healthy controls, n=88) are set out in Table 6.

TABLE 6
Sensitivity and specificity of the combination of biomarkers

| | AUC | Sensitivity | Specificity | PPV | NPV |
|---|---|---|---|---|---|
| SCREEN-HF | | | | | |
| vs Controls | | | | | |
| HF patients | 0.78 | 73.0% | 72.7% | 70.3% | 75.3% |
| vs Controls | | | | | |

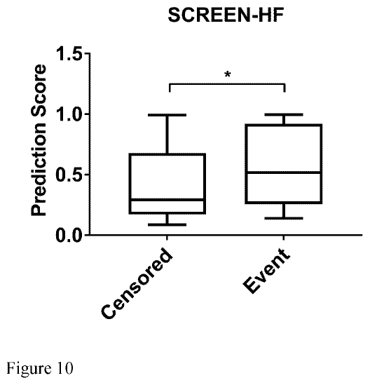

Prediction scores between study subjects who developed cardiovascular disease after enrolment in the study, and those who have no cardiovascular disease-related hospital admission are shown in FIG. 10.

Of the 99 participants in the SCREEN-HF cohort, 11 of them were admitted to hospital with cardiovascular diseases as the primary diagnosis. The prediction score generated by the three-marker panel in these 11 individuals ranged from 0.139 to 0.996 with a medium of 0.517 (IQR: 0.256-0.920), while in the individuals who did not have cardiovascular disease-related hospital admission, the prediction score ranged from 0.086 to 0.992 with a medium of 0.294 (IQR: 0.172-0.679). There is a statistical significant difference between the two groups of SCREEN-HF cohorts (p=0.0382).

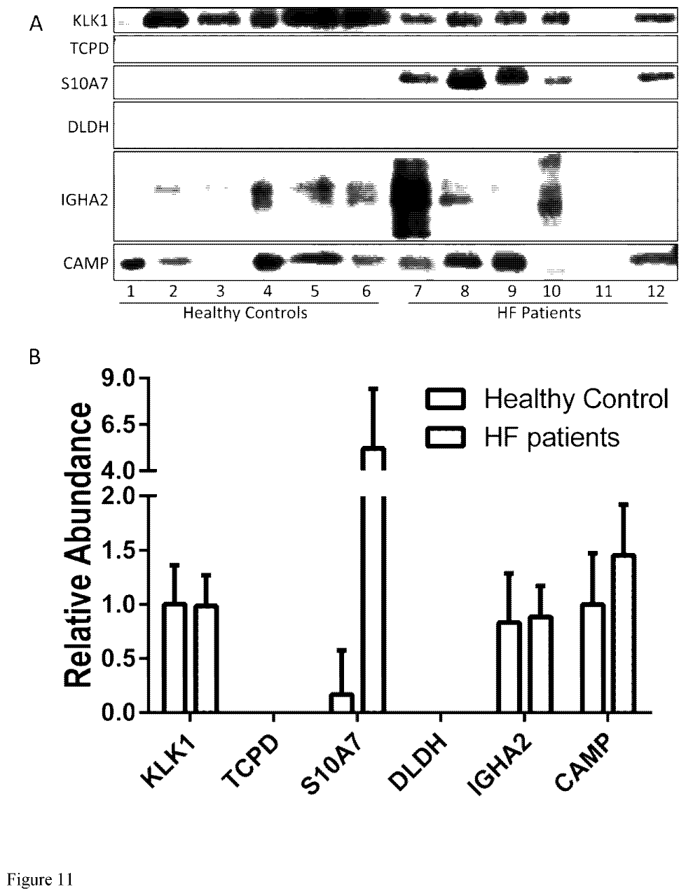

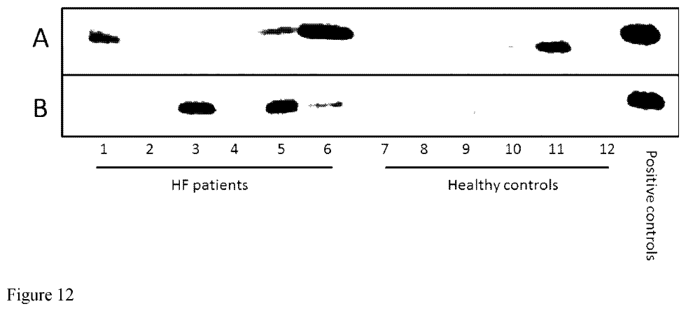

To validate KLK1, TCPD, S10A7, DLDH, IGHA2 and CAMP as members of a diagnostic panel, western blotting analysis was performed on 6 randomly chosen healthy control and 6 randomly chosen heart failure patients. As shown in FIG. 11, S10A7 and IGHA2 were detected in individual saliva samples. S10A7 was detected in 5 of the 6 heart failure patients' samples and only 1 of the 6 healthy control samples. Band intensity of each sample was normalized against the average band intensity of the healthy controls. Similar to the results from SWATH-MS, both S10A7 and IGHA2 demonstrated higher protein abundance in the heart failure patient samples compared to in the healthy control samples. The average band intensity of S10A7 in heart failure patients was 6 times higher than it was in the healthy control samples. IGHA2 has a higher abundance in heart failure patient samples compared to healthy control samples (1.06:1) but no significant different was observed. In contrast to findings in the initial screening, the expression of KLK1 in healthy control and patient samples was similar (1:0.98). CAMP expression was also different, with higher expression in heart failure patients than control (1:1.452). TCPD and DLDH were not detected with western blotting.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

### CITATION LIST

Australian Institute of Health and Welfare 2011. Cardiovascular disease: Australian facts 2011. Cardiovascular disease series. Cat. no. CVD 53. Canberra: AIHW. (http://www.aihw.gov.au/WorkArea/DownloadAssetaspx?id=10737418530)
Bailey U M and Schulz B L, Deglycosylation systematically improves N-glycoprotein identification in liquid chromatography-tandem mass spectrometry proteomics for analysis of cell wall stress responses in Saccharomyces cerevisiae lacking aAlg3p, J Chromatogr B Analyt Technol Biomed Life Sci, 2013; 923-924:16-21
Carlson K J, Lee D C S, Goroll A H, Leahy M and Johnson R A, An analysis of physicians' reasons for prescribing long-term digitalis therapy in outpatients, J Chron Dis, 1985; 38:733-739
Castagnola M, Inzitari R, Fanali C, Iavarone F, Vitali A, Desiderio C, Vento G, Tirone C, Romagnoli C, Cabras T, Manconi B, Sanna M T, Boi R, Pisano E, Olianas A, Pellegrini M, Nemolato S, Heizmann C W, Faa G and Messana I, The surprising composition of the salivary proteome of preterm human newborn, Mol Cell Proteomics, 2011; 10(1):M110.003467
Chang C Y, Picotti P, Hatenhain R, Heinzelmann-Schwarz V, Jovanovic M, Aebersold R and Vitek O, Protein significance analysis in selected reaction monitoring (SRM) measurements, Mol Cell Proteomics, 2012; 11(4):M111.014662
Clough T, Thaminy S, Ragg S, Aebersold R and Vitek O, Statistical protein quantification and significance analysis in label-free LC-MS experiments with complex designs, BMC Bioinformatics, 2012; 13(Suppl 16):S6

Foo J Y Y, Wan Y, Schulz B L, Kostner K, Atherton J, Cooper-White J, Dimeski G and Punyadeera C, Circulating fragments of N-terminal pro-B-type natriuretic peptides in plasma of heart failure patients, Clin Chem, 2013; 59:1523-1531
Harlan W R, Oberman A, Grimm R and Rosati R A, Chronic congestive heart failure in coronary artery disease: clinical criteria, Ann Intern Med, 1977; 86(2):133-138
Helmerhorst E J and Oppenheim F G, Saliva: a dynamic proteome, J Dent Res, 2007; 86:680-693
Krum H, Jelinek M V, Stewart S, Sindone A and Atherton J J, 2011 Update to national heart foundation of Australia and cardiac society of Australia and New Zealand guidelines for the prevention, detection and management of chronic heart failure in Australia, 2006, Med J Aust, 2011; 194(8):405-409
Loo J A, Yan W, Ramachandran P and Wong D T, Comparative human salivary and plasma proteomes, J Dent Res, 2010; 89:1016-1023
McKee P A, Castelli W P, McNamara P M and Kannel W B, The natural history of congestive heart failure: the Framingham study, N Engl J Med, 1971; 285(26):1441-1446
Marian A J and Nambi V, Biomarkers of cardiac disease, Expert Rev Mol Diagn, 2004; 4:805-20
Martinet W, Schrijvers D M, De Meyer G R Y, Herman A G and Kockx M M, Western array analysis of human atherosclerotic plaques: Downregulation of apoptosis-linked gene 2, Cardiovasc Res, 2003; 60(2):259-267
Ovchinnikov D A, Cooper M A, Pandit P, Coman W B, Cooper-White J J, Keith P, Wolvetang E J, Slowey P D and Punyadeera C, Tumor-suppressor gene promoter hypermethylation in saliva of head and neck cancer patients, Transl Oncol, 2012; 5(5):321-326
Palazzuoli A, Iovine F, Gallotta M and Nuti R, Emerging cardiac markers in coronary disease: Role of brain natriuretic peptide and other biomarkers, Minerva Cardioangiol, 2007; 55(4):491-496
Punyadeera C, Dimeski G, Kostner K, Beyerlein P and Cooper-White J, One-step homogeneous C-reactive protein assay for saliva, J Immunol Methods, 2011; 373:19-25
R Development Core Team (2011), R: A language and environment for statistical computing, Vienna, Austria: the R Foundation for Statistical Computing
Shamsham F and Mitchell J, Essentials of the diagnosis of heart failure, Am Fam Physician, 2000; 61(5):1319-1328
The Criteria Committee of the New York Heart Association, Nomenclature and Criteria for Diagnosis of Diseases of the Heart and Great Vessels, 9th ed., Little, Brown; Boston, 1994, pp. 253-256

1. A method for detecting early stage heart failure in a subject, the method comprising analysing a biological sample obtained from the subject and determining the concentration of at least one biomarker in the sample and assigning a heart failure classification to the subject if the concentration of the at least one biomarker is either higher or lower than a predefined reference concentration of the at least one biomarker.

2. The method of claim 1, wherein the predefined reference concentration of the at least one biomarker is determined from a biological sample taken from a healthy subject.

3. A method of detecting early stage heart failure in a subject, the method comprising analysing a biological sample obtained from the subject and determining the concentration of at least one biomarker in the sample, determining the concentration of the at least one biomarker in a biological sample obtained from a healthy subject, and assigning a heart failure classification to the subject if the concentration of the at least one biomarker in the sample from the subject is either higher or lower than the concentration of the at least one biomarker in the biological sample obtained from the healthy subject.

4. A method of screening for early stage heart failure in a subject, the method comprising analysing a biological sample obtained from the subject and determining the concentration of at least one biomarker in the sample and assigning a heart failure classification to the subject if the concentration of the at least one biomarker is either higher or lower than a predefined reference concentration of the at least one biomarker.

5. The method of any one of claims 1 to 4, wherein the at least one biomarker is selected from the group of proteins consisting of KLK1, TCPD, S10A7, DLDH, IGHA2, CAMP, KV110, NAMPT, COPB, SPR2A and HV311.

6. The method of claim 5, wherein the at least one biomarker is selected from the group of proteins consisting of KLK1, TCPD, S10A7, DLDH, IGHA2 and CAMP.

7. The method of claim 6, wherein the at least one biomarker is a biomarker panel comprising two, three, four, five or six of the proteins.

8. The method of claim 7, wherein the biomarker panel comprises three of the proteins.

9. The method of claim 8, wherein the biomarker panel comprises KLK1, S10A7, and CAMP.

10. The method of claim 5, wherein the at least one biomarker is selected from the group of proteins consisting of KV110, NAMPT, COPB, SPR2A and HV311.

11. The method of claim 10, wherein the at least one biomarker is a biomarker panel comprising two, three, four or five of the proteins.

12. The method of any one of claims 5 to 11, wherein the biological sample is selected from the group consisting of whole blood, serum, plasma, sputum or saliva.

13. The method of claim 12, wherein the biological sample is saliva.

14. A kit for detecting the presence of at least one biomarker associated with early stage heart failure, the kit comprising a solid support having immobilized thereon at least one molecule that specifically binds to the at least one biomarker.

15. A kit for detecting the presence of at least one biomarker associated with early stage heart failure, wherein the at least one biomarker is selected from the group consisting of KLK1, TCPD, S10A7, DLDH, IGHA2, CAMP, KV110, NAMPT, COPB, SPR2A and HV311, the kit comprising a solid support having immobilized thereon at least one molecule that specifically binds to the at least one biomarker.

16. The kit of claim 14 or claim 15, wherein the at least one molecule that specifically binds to the at least one biomarker is an antibody that specifically binds to the at least one biomarker.

17. The kit of claim 16, wherein the solid support has two, three, four, five or six antibodies immobilized thereon.

18. The kit of claim 17, wherein the solid support has three antibodies immobilized thereon.

19. The kit of claim 18, wherein the antibodies are antibodies to KLK1, S10A7, and CAMP.
